(19) United States
(12) Patent Application Publication
(10) Pub. No.: US 2011/0091537 A1
(43) Pub. Date: Apr. 21, 2011

(54) Anti-misuse solid oral pharmaceutical form provided with a specific modified release profile

(75) Inventors: Catherine Castan, Orlienas (FR); Anne-Sophie DAVIAUD-VENET, Saint-Genis-Laval (FR)

(73) Assignee: Flamel Technologies, Venissieux (FR)

(21) Appl. No.: 12/905,387

(22) Filed: Oct. 15, 2010

Related U.S. Application Data
(60) Provisional application No. 61/252,317, filed on Oct. 16, 2009.

(30) Foreign Application Priority Data
Oct. 16, 2009 (FR) 09 57270
Oct. 15, 2010 (IB) PCT/IB2010/054674

(51) Int. Cl. A61K 9/14; A61K 31/485; A61K 9/20; A61K 9/48; A61K 31/137

(52) U.S. Cl. 424/452; 424/490; 424/495; 424/494; 424/497; 514/282; 424/465; 514/646

(57) ABSTRACT

The present invention relates to a solid oral pharmaceutical form, with modified release of at least one active ingredient, containing at least microparticles containing said active ingredient and at least one viscosifying agent in a form isolated from said microparticles of active ingredient, characterized in that said microparticles possess an average diameter ranging from 100 to 600 μm, and are formed by a core containing at least said active ingredient and coated with at least one coating layer,
said core being formed by a support particle covered by a layer comprising at least said active ingredient,
said coating layer being formed by a material composed of at least 25 to 70% by weight relative to the total weight of said coating, of at least one polymer A insoluble in water, 30 to 75% by weight relative to the total weight of said coating, of at least one polymer B insoluble in water below pH 5 and soluble in water above pH 7, and 0 to 25% by weight relative to the total weight of said coating, of at least one plasticizer,
said polymers A and B being in a polymer(s) B/polymer(s) A weight ratio comprised between 0.25 and 4, and
said coating layer representing at least 35% by weight, relative to the total weight of said microparticle.

The present invention aims to propose a solid oral pharmaceutical form containing at least one viscosifying agent and an active ingredient formulated in the state of microparticles, the latter being resistant to crushing, in order to avoid misuse and suitable for obtaining a specific modified release profile comprising several release phases at least one of which depends on the pH.

Generally, the standard solid oral pharmaceutical forms, gelatin capsules or tablets, offer insufficient resistance to extraction of the active ingredient that they contain, and can therefore be subject to misuse.

Thus, a certain number of medicaments exist which are subject to abuse i.e. their use is diverted from the indication for which they have been authorized in order, on the contrary, to be used for purposes of obtaining a euphoriant effect, comparable to that obtained with illegal drugs. Narcotics can in particular be mentioned as examples of these medicaments.

The medicamentous solid galenic forms more particularly concerned by this misuse are the sustained release forms through which abusers can have a higher dose of active ingredient per tablet or gelatin capsule. It is generally sufficient to crush the gelatin capsule or the tablet in order to “break” the sustained release effect and transform the sustained release product into an immediate release product. Subsequently, the powder obtained can be either inhaled or swallowed, or also subjected to other liquid extraction methods in order to prepare an injectable (extraction in small volumes of solvents which are suitable for intravenous injection) or drinkable liquid (powder mixed with an alcoholic or non-alcoholic drink).

In order to prevent this kind of behaviour, it appears essential to be able to have solid oral pharmaceutical forms, not compatible with any use other than therapeutic use or uses officially approved by the competent public health authorities.

Anti-misuse means have already been proposed for pharmaceutical forms. For example, the document WO 2007/054378 proposes solid oral pharmaceutical forms constituted by sustained-release microparticles of active ingredient which are resistant to crushing. These microparticles can also be combined with a viscosifying agent and/or a sequestering agent.

However, the microparticles described in this document possess a coating which is insensitive to pH, and can therefore release the active ingredient that they contain only in a, continuous and regular sustained manner. As a result, the pharmaceutical forms described in WO 2007/054378 prove unsuitable for obtaining a specific modified release profile comprising several release phases at least one of which depends on the pH.

Now, certain active ingredients or therapeutic objectives require very different release profiles, comprising different release phases certain of which depend on the pH. This makes it possible in particular to cause the release of the active ingredient to coincide with targeted zones in the small intestine and thus optimize the action profile of the active ingredient.

The present invention aims precisely to remedy this defect, by proposing solid oral pharmaceutical forms with anti-misuse properties, which are in parallel suitable for obtaining a modified release profile comprising several release phases at least one of which depends on the pH.

More precisely, the present invention relates to a solid oral pharmaceutical form, with modified release of at least one active ingredient, containing at least one viscosifying agent and microparticles containing said active ingredient, and characterized in that said microparticles possess an average diameter ranging from 100 to 600 μm, and are formed by a core containing at least said active ingredient and coated with at least one coating layer,
said core being formed by a support particle covered by a layer comprising at least said active ingredient, and
said coating layer being formed by a material composed of at least:
   25 to 70% by weight relative to the total weight of said coating, of at least one polymer A which is insoluble in water,
   30 to 75% by weight relative to the total weight of said coating, of at least one polymer B which is insoluble in water below pH 5 and soluble in water above pH 7, and
   0 to 25% by weight relative to the total weight of said coating, of at least one plasticizer,
said coating layer representing at least 35% by weight, relative to the total weight of said microparticle.

In particular, the coating of the microparticles according to the invention comprises the two polymers A and B in a polymer(s) B/polymer(s) A weight ratio comprised between 0.25 and 4.

As is apparent from the foregoing, a solid oral form according to the invention comprises at least one viscosifying agent which is in a form isolated from said microparticles containing said active ingredient. In other words, it is not present in the core of said microparticles, nor is it in their coating.

Thus said viscosifying agent and said microparticles of active ingredient constitute two distinct entities, both of them contained in the solid oral pharmaceutical form of the invention.

Preferably, the solid oral form according to the invention comprises viscosifying agent(s) only in a form isolated from said microparticles of active ingredient.

Within the meaning of the invention by “solid oral pharmaceutical form”, is generally meant tablets, powders, gelatin capsules or other analogous products intended for administration by oral route in humans or for veterinary use.

In view of their specificities, the solid oral forms according to the invention are advantageously provided with effective anti-misuse properties.

Within the meaning of the invention by “form provided with anti-misuse properties”, is generally meant a pharmaceutical form the physico-chemical properties of which are such that the use of the medicament for purposes other than those authorized, is made very difficult.

More particularly, as is apparent from the examples presented hereafter, the microparticles of the solid oral form according to the invention prove particularly resistant to crushing, so that it is very difficult to break their coating and by this means access the active ingredient in an immediately absorbable form.

By the expression “resistant to crushing”, is meant that the microparticles according to the invention are such that they make it possible, in the event of crushing, to maintain the specific modified release profile for at least 40%, preferably at least 60%, and still more preferentially at least 80% of the microparticles with modified-release of active ingredient. The crushing envisaged here can be for example any crushing carried out according to the techniques usually implemented by those responsible for misuse, such as for example: pestle and mortar, coffee mill, crushing between two spoons, crunching/chewing, etc.

A crushing test usable to measure the resistance to crushing and lying on the pestle and mortar technique is described in detail hereafter and used in examples.

More particularly, the resistance to crushing of the microparticles can be measured according to the following protocol.

A dose of active ingredient in the form of microparticles or in an intact oral pharmaceutical form (a tablet or the content of a gelatin capsule) is introduced into a 250 ml pyrex mortar and crushed using the pyrex pestle corresponding to the mortar for 50 revolutions, i.e. for approximately 1 minute.

Regarding the release profile, it can be characterized by a dissolution test. This test is carried out according to the method of the European Pharmacopoeia 6^{th} Edition, 6.5 Chapter 2.9.3—Test for dissolution of the solid forms. As a comparison, dissolution profiles of intact and crushed microparticles or intact and crushed oral pharmaceutical form can be carried out. In particular, these profiles can be compared after putting into contact for 30 minutes the crushed powder and the intact formulation with the appropriate dissolution medium.

A solid form according to the invention, can be transformed neither to a dry form which can be administered by nasal aspiration and with immediate release of the active ingredient, nor to an injectable form with immediate release of the active ingredient, and is not suitable for extraction of the active ingredient by chewing and/or crushing.

Similarly, when the intact or crushed solid oral form is introduced into a small volume of injectable solvent, the viscosifying agent present in the solid oral form of the invention will transform the mixture to a non-homogeneous paste which is too viscous to be filtered or transferred into a syringe, thus making it impossible to obtain an injectable liquid containing the active ingredient in an immediately available form.

Within the meaning of the invention, by the expression “with modified release” is meant to describe the ability of the microparticles considered according to the invention to exhibit at least in vitro, a release profile of the combined active ingredient which comprises three phases, and the different sequences of which are triggered according to two distinct mechanisms which are independent of each other, one being activated by time (1^{st} mechanism) and the other activated by pH (2^{nd} mechanism).

As explained hereafter, this ability of the microparticles according to the invention depends on the specificities of their coating.

FIG. 1 is a diagrammatic representation of the dissolution profile expected for a solid form according to the invention when said form is exposed successively to
an aqueous acid medium with a pH of less than 4, representative of the pH conditions encountered in the stomach, and
an aqueous medium at a pH of greater than 7, representative of the pH conditions encountered in the small intestine.

In the acid aqueous medium at a pH of less than 4, it is the 1^{st} mechanism which acts. This is time-activated. According to this 1^{st} mechanism, the release of the active ingredient is triggered (phase 2) after a determined contact time of the solid form with this aqueous medium (phase 1). The delayed and sustained release profile can be observed with a given latency period of less than 12 hours, in particular between 0.5 and 8 hours, or even between 1 and 5 hours. The latency period corresponds to the time below which the microparticles release less than 10% of their dose of active ingredient(s).

On the other hand, when these same microparticles, having remained in the acid aqueous medium at a pH of less than 4, come into contact with the aqueous medium at a pH of greater than 7, it is the 2^{nd} mechanism which acts. This is pH-activated. In this case, the release of the active ingredient is accelerated once the microparticles come into contact with this aqueous medium (phase 3).

These two release mechanisms of the active ingredient or active ingredients formulated in the solid form according to the invention are therefore generally ensured in sequence. In other words, the transition from the first phase to the second phase is triggered in vivo by a contact time with the acid medium in the stomach, whereas the transition from the second phase to the third phase is triggered by the change in pH encountered when the microparticles leave the stomach to enter the intestine.

It is understood that these three phases occur in the abovementioned sequential order when the increase in pH occurs after initiation of phase 2. In the event of the increase in pH, which triggers the 2^{nd} mechanism, occurring during or at the end of phase 1, phase 3 would then occur early.

It should be noted that this modified release profile differs from the release profiles obtained with enteric coatings, which have only one single release mechanism triggered by the pH, resulting in negligible release while the form remains in an acid medium. Thus, the enteric coatings do not allow the release of the active ingredient in the stomach.

According to a particular embodiment, the solid oral form according to the invention can also comprise at least one sequestering agent as described more precisely hereafter.

According to another particular embodiment, the solid oral form according to the invention can also comprise one or more excipients distinct from the modified-release microparticles.

The present invention proves more particularly advantageous with regard to active substances, indiscriminately referred to as “active ingredients”, in particular pharmaceutical or veterinary substances, the abuse of which can give rise to addictive behaviour, such as for example those classified within the category of stupefacients, narcotics or analgesics. For obvious reasons, it is not however limited to the use of this type of active ingredient.

### Microparticle System

The solid oral pharmaceutical form according to the invention comprises modified-release microparticles the composition and architecture of which are adjusted, on the one hand, to render them resistant to crushing and on the other hand, to confer the specific modified release profile sought for the active ingredient or mixture of active ingredients that they contain.

As specified previously, the microparticles into consideration according to the invention possess an average diameter ranging from 100 to 600 μm.

Preferably, the microparticles possess an average diameter ranging from 150 to 350 μm, more particularly 200 to 300 μm, in particular 250 to 300 μm.

The average diameter is determined by laser diffraction.

Generally, the use of the laser diffraction method, in particular as explained in the Pharmacopoeia 6th Edition, Chapter 2.9.31., to characterize a size by volume mean diameter, is preferred up to a size scale of 700 μm.

More particularly, the equivalent volume mean diameter of the microparticles according to the invention, written D(4;3), can be obtained according to the following measuring protocol.

The size distribution of the particles is measured by laser diffraction using a Mastersizer® 2000 device from Malvern Instruments equipped with a dry powder sampler of Scirocco 2000 type. Starting from the particle-size distribution measured over a wide range, the equivalent volume mean diameter or D(4;3) is calculated according to the following formula:

D(4;3)=Σ(d⁴)/Σ(d³)

The microparticles into consideration according to the invention are structurally organized in a core, coated or film-coated with a coating. This structure is shown in FIG. 2.

### Core of the Microparticles

The core of the microparticles according to the invention has advantageously a compact and globally spherical shape.

The core of the microparticles according to the invention is more particularly a granule obtained by application of a layer formed wholly or partly by the active ingredient on a support particle.

Thus, the microparticles according to the invention, as represented diagrammatically in FIG. 2, each comprise a support particle, at least one active layer comprising the active ingredient(s) and covering the support particle, and at least one coating allowing the modified release of the active ingredient.

The support particles can be:
crystals or spheres of lactose, sucrose (such as for example Compressuc® PS from Tereos), microcrystalline cellulose (such as for example Avicel® from FMC Biopolymer, Cellet® from Pharmatrans or Celphere® from Asahi Kasei), sodium chloride, calcium carbonate (such as for example Omyapure® 35 from Omya), sodium hydrogen carbonate, dicalcium phosphate (such as for example Dicafos® AC 92-12 from Budenheim) or tricalcium phosphate (such as for example Tricafos® SC93-15 from Budenheim);
composite spheres or granules, for example spheres of sugar produced by granulation of sucrose with starch used as binding agent (such as for example Suglets® from NP Pharm), spheres of calcium carbonate produced with starch as binding agent (such as for example Destab® 90 S Ultra 250 from Particle Dynamics) or maltodextrin (Hubercal® CCG4100 from Huber).

The support particles can also be any other particles of pharmaceutically acceptable excipient(s) such as for example particles of hydroxypropyl cellulose (such as for example Klucel® from Aqualon), guar gum particles (such as for example Grinsted® Guar from Danisco), xanthan particles (such as for example Xantural® 180 from CPKelco).

Advantageously, the support particle has an average diameter less than or equal to 300 μm, preferably comprised between 50 and 250 μm, in particular between 70 and 150 μm.

According to a particular embodiment of the invention, the support particles are spheres of sugar or spheres of microcrystalline cellulose, such as for example Cellet® 90 marketed by Pharmatrans and the volume mean diameter of which is equal to approximately 95 μm, or also Celphere® SCP 100 and more particularly the fraction of Celphere® SCP 100 less than 100 μm after sieving on a 100 μm sieve and the volume mean diameter of which is approximately 100 μm, or also particles of dicalcium phosphate, for example Dicafos® AC 92-12 and more particularly the fraction of Dicafos® AC 92-12 comprised between 50 and 100 μm after sieving Dicafos® AC 92-12 on 50 μm and 100 μm sieves and the volume mean diameter of which is approximately 75 μm.

The active layer covering the support particle for forming the core of the microparticles of the invention can optionally comprise, besides the active ingredient(s), one or more binding agent selected from:
hydroxypropylcellulose (such as for example Klucel® EF from Aqualon-Hercules), hydroxy-propylmethylcellulose (or hypromellose) (such as for example Methocel® E3 or E5 from Dow), methylcellulose (such as for example Methocel® A15 from Dow),
polyvinylpyrrolidone (or povidone) (such as for example Plasdone® K29/32 from ISP or Kollidon® 30 from BASF), vinyl pyrrolidone and vinyl acetate copolymer (or copovidone) (such as for example Plasdone® S630 from ISP or Kollidon® VA 64 from BASF),
dextrose, pregelatinized starches, maltodextrin.

The preferred binding agents are povidone (Plasdone® K29/32 from ISP), hydroxypropylcellulose (Klucel® EF from Aqualon-Hercules) or hypromellose (Methocel® E3 or E5 from Dow).

The layer containing at least one active ingredient and covering the support particle represents at least 50% by weight, preferably at least 60% by weight, more preferably from 70 to 95% by weight and in particular from 80 to 90% by weight of the weight of the granule.

The active layer covering the support particle for forming the core of the microparticles of the invention can also optionally comprise, besides the active ingredient(s), one or more physiologically acceptable excipients, such as surfactants, disintegrators, fillers, agents controlling or modifying the pH (buffers), anti-foaming agents the choice and quantity adjustment of which is clearly within the competence of a person skilled in the art.

### Active Ingredients

As regards the active ingredient, for obvious reasons, it is clear that the microparticles into consideration according to the invention are compatible with a great diversity of active ingredients.

However, the solid forms according to the invention are particularly advantageous with respect to the utilization of active ingredients the abuse of which can give rise to addictive behaviour, such as for example those classified within the category of stupefacients, analgesics or narcotics.

Thus, the active ingredient contained in the coated microparticles according to the invention can be, for example, advantageously chosen from at least one of the following families of active ingredients: amphetamines, analgesics, anorexigens, antidepressants, antiepileptics, antiparkinsonians, anxiolytics, barbiturates, benzodiazepines, hypnotics, narcotics, neuroleptics, psychostimulants and psychotropics.

In the case where the active ingredient is a narcotic, it is preferably an opioid.

More precisely, the narcotic utilized can be chosen from oxycodone, oxymorphone, hydromorphone, hydrocodone, tramadol and their pharmaceutically acceptable salts.

### Coating of the Microparticles

Within the scope of the present invention, the core, containing the active ingredient or a mixture of active ingredients, is covered with a coating the composition and thickness of which are precisely adjusted in order on the one hand to obtain the specific release profile of the active ingredient in vitro in three phases triggered by two independent release mechanisms, one activated by time and the other activated by pH and on the other hand to contribute to render these modified-release microparticles resistant to crushing.

The coating which covers the core of the microparticles represents at least 35% by weight of the total weight of the modified-release microparticle, i.e. a coating level of at least 35%.

More particularly, the coating can represent from 35 to 60% by weight, in particular from 40 to 55% by weight, more particularly from 45 to 55% by weight of the total weight of the modified-release microparticle.

The coating can be formed by a composite material obtained by mixing:
at least one water-insoluble polymer A,
at least one second polymer B which is insoluble in water at a pH of less than 5 and soluble in water at a pH greater than 7;
and optionally at least one plasticizer.

The water-insoluble polymer A is more particularly chosen from ethylcellulose, for example marketed under the name Ethocel®, cellulose acetate butyrate, cellulose acetate, ammonio (meth)acrylate copolymers (ethyl acrylate, methyl methacrylate and trimethylammonio ethyl methacrylate copolymer) in particular those marketed under the names Eudragit® RL and Eudragit® RS, poly(meth)acrylic acid esters, in particular those marketed under the name Eudragit® NE and mixtures thereof.

The coating of the microparticles can contain from 25% to 70% by weight polymer(s) A relative to its total weight.

According to a preferred embodiment, the coating of the microparticles has a content of polymer(s) A comprised between 30 and 65%, in particular between 35 and 60% by weight, more particularly between 35 and 55% by weight, and still more particularly between 35 and 50% by weight, relative to its total weight.

By way of non-limitative illustration of polymers B which are suitable for the invention, i.e. insoluble in water at a pH of less than 5 and soluble in water at a pH greater than 7, there can in particular be mentioned:
methacrylic acid and methyl methacrylate copolymer(s),
methacrylic acid and ethyl acrylate copolymer(s),
cellulose acetate phthalate (CAP),
cellulose acetate succinate (CAS),
cellulose acetate trimellitate (CAT),
hydroxypropylmethylcellulose phthalate (or hypromellose phthalate) (HPMCP),
hydroxypropylmethylcellulose acetate succinate (or hypromellose acetate succinate) (HPMCAS),
carboxymethylethylcellulose,
shellac gum,
polyvinyl acetate phthalate (PVAP),
and mixtures thereof.

According to a preferred embodiment of the invention, this polymer B is chosen from the methacrylic acid and methyl methacrylate copolymer(s), the methacrylic acid and ethyl acrylate copolymer(s) and mixtures thereof.

The polymers B dissolve in water at a given pH value, comprised between 5 and 7, this value varying as a function of their intrinsic physico-chemical characteristics, such as their chemical nature and their chain length.

For example, the polymer B can be a polymer the solubilization pH value of which is:
5.0, such as for example hydroxypropylmethylcellulose phthalate and in particular that marketed under the name HP-50 by Shin-Etsu,
5.5, such as for example hydroxypropylmethylcellulose phthalate and in particular that marketed under the name HP-55 by Shin-Etsu or methacrylic acid and ethyl acrylate copolymer 1:1 and in particular that marketed under the name Eudragit L100-55 by Evonik,
6.0 such as for example a methacrylic acid and methyl methacrylate copolymer 1:1 and in particular that marketed under the name Eudragit L100 by Evonik,
7.0 such as for example a methacrylic acid and methyl methacrylate copolymer 1:2 and in particular that marketed under the name Eudragit S100 by Evonik.

All of these polymers are soluble at a pH value above their solubilization pH.

The coating is advantageously composed of at least 30 to 75%, in particular 30 to 70%, in particular 35 to 65%, or even 35 to 60% by weight polymer(s) B relative to its total weight.

The coating of the microparticles according to the invention comprises the two polymers A and B in a polymer(s) B/polymer(s) A weight ratio greater than 0.25, in particular greater than or equal to 0.3, in particular greater than or equal to 0.4, in particular greater than or equal to 0.5, or even greater than or equal to 0.75.

According to another embodiment variant, the polymer(s) B/polymer(s) A ratio is moreover less than 8, in particular less than 5, notably less than 4, or even less than 2 and more particularly less than 1.5.

The polymer(s) B/polymer(s) A weight ratio can be comprised between 0.25 and 8. However, it is advantageously comprised between 0.25 and 5, in particular between 0.3 and 4, more particularly between 0.4 and 2, notably between 0.5 and 2, and more particularly between 0.75 and 1.5.

Preferentially, the coating of the microparticles according to the invention comprises the two polymers A and B in a polymer(s) B/polymer(s) A weight ratio comprised between 0.25 and 4.

According to a particular embodiment, the coating of the microparticles is formed by at least one mixture comprising, as polymer A, at least ethylcellulose or cellulose acetate butyrate or the ammonio (meth)acrylate copolymer or a mixture thereof, with, as polymer B, at least one methacrylic acid and ethyl acrylate copolymer or a methacrylic acid and methyl methacrylate copolymer or a mixture thereof.

Thus, according to a particular embodiment, the coating of the microparticles according to the invention can be advantageously formed by at least one polymer B/polymer A pair chosen from the following pairs:

1. methacrylic acid and ethyl acrylate copolymer, 1:1/ethylcellulose,

2. methacrylic acid and methyl methacrylate copolymer, 1:2/ethylcellulose,

3. mixture of methacrylic acid and ethyl acrylate copolymer, 1:1 and methacrylic acid and methyl methacrylate copolymer, 1:2/ethylcellulose,

4. methacrylic acid and ethyl acrylate copolymer, 1:1/cellulose acetate butyrate,

5. methacrylic acid and methyl methacrylate copolymer, 1:2/cellulose acetate butyrate,

6. mixture of methacrylic acid and ethyl acrylate copolymer, 1:1 and methacrylic acid and methyl methacrylate copolymer, 1:2/cellulose acetate butyrate,

7. methacrylic acid and ethyl acrylate copolymer, 1:1/cellulose acetate,

8. methacrylic acid and methyl methacrylate copolymer, 1:2/cellulose acetate,

9. mixture of methacrylic acid and ethyl acrylate copolymer, 1:1 and methacrylic acid and methyl methacrylate copolymer, 1:2/cellulose acetate,

10. methacrylic acid and ethyl acrylate copolymer 1:1/ammonio (meth)acrylate copolymer,

11. methacrylic acid and methyl methacrylate copolymer 1:2/ammonio (meth)acrylate copolymer, and

12. mixture of methacrylic acid and ethyl acrylate copolymer 1:1 and methacrylic acid and methyl methacrylate copolymer 1:2/ammonio (meth)acrylate copolymer.

According to a particularly preferred embodiment, the coating comprises at least the pair polymer B/polymer A, polymer B being formed by the mixture of methacrylic acid and ethyl acrylate copolymer 1:1 and methacrylic acid and methyl methacrylate copolymer 1:2, and polymer A being ethylcellulose.

The coating of the microparticles according to the invention can also comprise at least one plasticizer.

This plasticizer can in particular be chosen from:
glycerol and its esters, and preferably from the acetylated glycerides, glyceryl-mono-stearate, glyceryl-triacetate, glyceryl-tributyrate,
the phthalates, and preferably from dibutyl phthalate, diethyl phthalate, dimethyl phthalate, dioctyl phthalate,
the citrates, and preferably from acetyl tributylcitrate, acetyl triethyl citrate, tributyl citrate, triethyl citrate,
the sebacates, and preferably from diethyl sebacate, dibutyl sebacate,
the adipates,
the azelates,
the benzoates,
chlorobutanol,
the polyethylene glycols,
the vegetable oils,
the fumarates, preferably diethyl fumarate,
the malates, preferably diethyl malate,
the oxalates, preferably diethyl oxalate,
the succinates; preferably dibutyl succinate,
the butyrates,
the cetyl alcohol esters,
the malonates, preferably diethyl malonate,
castor oil,
and mixtures thereof.

In particular, the coating can comprise less than 25% by weight, preferably 5% to 20% by weight, and, still more preferably, 10% to 20% by weight of plasticizer(s) relative to its total weight.

Thus, the coating of particles according to the invention can be advantageously formed by at least:
30 to 60%, in particular 35 to 55% by weight, at least one polymer A chosen from ethylcellulose, cellulose acetate butyrate, cellulose acetate, an ammonio (meth)acrylate copolymer or a mixture thereof,
30 to 70%, in particular 30 to 60% by weight, at least one polymer B chosen from a methacrylic acid and methyl methacrylate copolymer, in particular a methacrylic acid and methyl methacrylate copolymer 1:1 or a methacrylic acid and methyl methacrylate copolymer 1:2; a methacrylic acid and ethyl acrylate copolymer, in particular a methacrylic acid and ethyl acrylate copolymer 1:1 or a methacrylic acid and ethyl acrylate copolymer 1:2, and mixtures thereof,
and 10 to 20% by weight at least one plasticizer such as for example triethyl citrate or polyethylene glycol.

As a non-limitative illustration of the particles according to the invention, there can in particular be mentioned those the coating of which possesses one of the following compositions.
35 to 55% ethylcellulose
30 to 60% of a mixture of methacrylic acid and ethyl acrylate copolymer 1:1 and methacrylic acid and methyl methacrylate copolymer 1:2
10 to 20% triethyl citrate
35 to 55% cellulose acetate butyrate
30 to 60% methacrylic acid and ethyl acrylate copolymer 1:1
10 to 20% triethyl citrate
35 to 55% ethylcellulose
30 to 60% of a mixture of methacrylic acid and ethyl acrylate copolymer 1:1 and methacrylic acid and methyl methacrylate copolymer 1:2
10 to 20% polyethylene glycol

Of course, the coating can comprise various other additional adjuvants used in a standard manner in the field of coating. These can be, for example:
pigments and colouring agents, such as titanium dioxide, calcium sulphate, precipitated calcium carbonate, iron oxides, natural food colouring agents such as caramels, carotenoids, carmine, the chlorophyllins, Rocou (or annatto), the xanthophylls, the anthocyans, betanin, aluminium and synthetic food colouring agents such as the yellows No. 5 and No. 6, the reds No. 3 and No. 40, the green No. 3 and Emerald green, the blues No. 1 and No. 2;
fillers, such as talc, magnesium stearate, magnesium silicate:
anti-foaming agents, such as simethicone, dimethicone;
surfactants, such as the phospholipids, polysorbates, polyoxyethylene stearates, fatty acid esters and polyoxyethylenated sorbitol, polyoxyethylenated hydrogenated castor oils, polyoxyethylenated alkyl ethers, glycerol monooleate,
and mixtures thereof.

According to a particular embodiment of the invention, the coating of the microparticles according to the invention contains no active ingredient.

According to another embodiment of the invention, the coating contains no compound soluble at a pH value ranging from 1 to 4.

The coating can be single or multi-layer. According to an embodiment variant, it is made up of a single layer formed by the composite material defined previously.

According to a particularly preferred embodiment of the invention, the coating comprises less than 30% by weight, relating to the total weight, of lubricating agent(s), in particular less than 20%, notably less than 10%, more particularly less than 5% by weight, and is even advantageously totally free of lubricating agent.

Within the meaning of the invention a lubricating agent, also named “sliding agent”, is a substance used to decrease the aggregation of polymer within the coating phase of microparticles.

In particular, the coating of microparticles according to the invention comprises, as such, less than 30% by weight of talc, relating to the total weight, in particular less than 20%, notably less than 10%, more particularly less than 5% by weight, and is even advantageously totally free of talc.

### Viscosifying Agent

As specified previously, a solid oral form according to the invention also comprises at least one viscosifying agent, intended to reinforce the prevention of intentional misuse of the active ingredient contained in the solid oral form.

More precisely, it has the objective, when the solid oral form is brought into contact with a small volume of injectable solvent, of transforming the corresponding mixture into a non-homogeneous paste, which is too viscous to be filtered or transferred into a syringe, thus making it impossible to obtain an injectable liquid containing the active ingredient in an immediately available form.

Within the meaning of the invention, an oral solid form according to the invention comprises therefore at least one viscosifying agent in a form isolated from the microparticles of active ingredient.

Preferentially, the oral solid form according to the invention comprises only viscosifying agent in a form isolated from the microparticles of active ingredient.

According to a preferred embodiment, the viscosifying agent is chosen from the viscosifying agents which are soluble in at least one of the solvents chosen from water, alcohols, ketones and mixtures thereof.

According to a preferred embodiment, the viscosifying agent is capable of increasing the viscosity of a small volume (between 2.5 ml and 10 ml) of solvent, in order to prevent injection by intra-venous route. In fact, the viscosity becomes so high that the drawing off of the mixture formed by the utilization of the solid oral form according to the invention in a small volume of injectable solvent by a syringe becomes impossible.

According to a particular embodiment, a solid form according to the invention can advantageously comprise a mixture of several viscosifying agents which will be effective both in the case of an extraction in aqueous phase and in an organic solvent.

As regards the quantity of viscosifying agent, it can easily be determined by a person skilled in the art. This quantity advantageously corresponds to the minimum quantity necessary to bring the viscosity of 2.5 ml of extraction liquid to a value equal to or greater than 100 mPa·s, preferably 200 mPa·s, and still more preferably above 500 mPa·s, and better still 1000 Pa·s.

According to a particular embodiment, the viscosifying agent is chosen from:
the polyacrylic acids, in particular the carbomers, for example Carbopol®,
the polyalkylene glycols, for example the polyethylene glycols,
the polyalkylene oxides, for example the polyethylene oxides or polyoxyethylene,
the polyvinylpyrrolidones,
the gelatins,
the polysaccharides, preferably chosen from sodium alginate, the pectins, guar gum, the xanthans, the carrageenans, the gellans, hydroxypropylcellulose, hydroxypropylmethylcellulose, methylcellulose, hydroxyethylcellulose and carboxymethylcellulose,
and mixtures thereof.

According to a particularly preferred embodiment, the viscosifying agent is a polyoxyethylene, in particular a polyoxyethylene possessing a high molecular weight, and more particularly a polyoxyethylene having an average molecular weight ranging from 1 million g/mole to approximately 8 million g/mole.

As viscosifying agent, there can in particular be mentioned the polyoxyethylene marketed by Dow under the reference Sentry Polyox WSR® 303.

The viscosifying agent, for example the high molecular weight polyoxethylene, is in the form of microparticles, distinct from the microparticles with modified-release of active ingredient according to the invention as described previously.

Advantageously, the microparticles of viscosifying agent have a size distribution similar to that of the microparticles with modified-release of active ingredient according to the invention, so that they cannot be separated from the microparticles of active ingredient by sieving.

Advantageously, the volume mean diameter of the microparticles of viscosifying agent is comprised between 0.5 and two times, preferably comprised between 0.7 and 1.5 times, still more preferably comprised between 0.8 and 1.25 times the volume mean diameter of the microparticles with modified-release of active ingredient.

### Sequestering Agent

According to another particular embodiment, the solid oral form according to the invention can also comprise at least one sequestering agent.

The sequestering agent will in particular make it possible to capture the active ingredient which could be extracted from the microparticles of the invention, after remaining for several hours in a drink, and thus make it unavailable for immediate absorption.

More particularly, the sequestering agent is an ionic compound, capable of forming in solution, for example in an aqueous or alcoholic drink, a complex with the active ingredient itself in the ionized form, and in particular a slightly soluble complex.

Thus, when the active ingredient and the sequestering agent are to be found simultaneously in a suitable solvent, for example in the case of an illicit attempt at extraction of the active ingredient, the sequestering agent is capable of inducing a complexing or a chemical interaction with the active ingredient in said solvent. Within the meaning of the present invention, a suitable solvent is a usual solvent chosen from water and aqueous solutions, such as the water-ethanol mixtures, alcohol, alcoholic drinks, sodas, vinegar, hydrogen peroxide, and mixtures thereof.

The sequestering agents used to trap the active ingredient are harmless, including for regular use. These are pharmacologically inert products approved by the different pharmacopoeias and drug registration authorities.

If the sequestering agent is present, it is in the form of microparticles distinct from the microparticles with modified-release of active ingredient.

According to a particular embodiment, the sequestering agent comprises a salt, which contains ions capable of forming a complex with the active ingredient in solution. If, in solution, the active ingredient is in cationic form, the sequestering agent is an anionic compound. In the same way, when the active ingredient in solution is in anionic form, the sequestering agent is a cationic compound.

In non-limitative manner, among the anionic sequestering agents, the following compounds can be mentioned:
sodium dodecyl sulphate, sodium docusate;
anionic polymers, such as the cross-linked polyacrylic acids (or carbomer for example Carbopol®), carboxymethycellulose salts such as sodium carmellose or calcium carmellose, cross-linked carboxymethylcellulose such as sodium croscarmellose and its derivatives, polysaccharides, for example the alginates, xanthan gum or gum arabic, propylene glycol alginate-(sulphonate);
mono- or polyvalent salts, such as the glucuronates, citrates, acetates, carbonates, gluconates, succinates, phosphates, glycerophosphates, lactates, trisilicates, fumarates, adipates, benzoates, salicylates, tartrates, sulphonamides, acesulphames;
saponified fatty acids, such as stearic acid salts, palmitic acid salts;
polyamino acids, proteins or anionic peptides, such as the glutamates, aspartates, albumins, caseines, globulins;
strongly acid cation exchange resins, such as the sulfonated copolymers of styrene and divinylbenzene, such as for example Amberlite® IRP69, Amberlite® IR69F, Amberlite® 200 or Amberlite® 200C, marketed by Rohm and Haas, or Dowex® 88, marketed by Dow;
weakly acid cation exchange resins, such as the cross-linked copolymers of methacrylic acid and divinylbenzene or their salts, such as for example Amberlite® IRP88 and Amberlite IRP64, marketed by Rohm and Haas or WEX MAC-3® marketed by Dow;
and mixtures thereof.

Among the cationic sequestering agents, there can be mentioned:
quaternary ammonium salts, such as tetradecyl trimethyl ammonium bromide or benzethonium chloride;
cationic polymers, such as the chitosans and the ethyl acrylate, methyl methacrylate and trimethylammonioethyl methacrylate chloride copolymers (for example, Eudragit® RS, Eudragit® RL) and ethyl acrylate, methyl methacrylate and methacrylic acid copolymers (for example Eudragit® E);
cationic polyamino acids, proteins or peptides such as polylysines and polyarginines;
basic anion exchange resins, such as the phenolic polyamines such as Amberlite® IRP58, marketed by Rohm and Haas, the copolymers of styrene and divinylbenzene bearing quaternary ammonium functions, such as for example Duolite® AP143 or Amberlite® IRP67, marketed by Rohm and Haas, or DOWEX 22, marketed by Dow;
and mixtures thereof.

According to a particular embodiment of the invention, the sequestering agent is chosen from:
sodium dodecyl sulphate or sodium docusate;
quaternary ammonium salts, such as tetradecyl trimethyl ammonium bromide or benzethonium chloride;
strongly acid cation exchange resins, when the active ingredient in solution is cationic, or strongly basic anion exchange resins when the active ingredient in solution is anionic, according to the polarity of the active ingredient, and mixtures thereof.

According to a particularly preferred embodiment of the invention, when the active ingredient in solution is in cationic form, the sequestering agent is chosen from
strongly acid cation exchange resins, such as the sulfonated copolymers of styrene and divinylbenzene, such as for example Amberlite® IRP69, Amberlite® IR69F, Amberlite® 200 or Amberlite® 200C, marketed by Rohm and Haas, or Dowex® 88, marketed by Dow; and
weakly acid cation exchange resins such as the cross-linked copolymers of methacrylic acid and divinylbenzene or their salts, such as for example Amberlite® IRP88 and Amberlite® IRP64, marketed by Rohm and Haas or WEX MAC-3® marketed by Dow.

The quantity of agent is adapted by a person skilled in the art by calculating the quantity of ionic charge necessary to trap all or part of the dose of active ingredient contained in the solid unitary form.

In particular, the quantity of sequestering agent must be such that it makes it possible to complex enough active ingredient so that the remaining quantity of free active ingredient in solution is not enough to obtain the desired effect in the case of illicit use.

In particular, the quantity of sequestering agent is such that it makes it possible to complex at least 40%, preferably at least 50%, still more preferably at least 60%, preferably at least 70% of the dose of active ingredient contained in the solid unitary form.

Preferably, the quantity of sequestering agent is enough to complex all the active ingredient in the unitary dose.

Advantageously, the microparticles of sequestering agent have a size distribution similar to that of the microparticles of active ingredient, so that they cannot be separated from the microparticles of active ingredient by sieving or sedimentation.

Preferably, the volume mean diameter of the microparticles of sequestering agent is comprised between 0.5 and two times, preferably comprised between 0.7 and 1.5 times, still more preferably comprised between 0.8 and 1.25 times the volume mean diameter of the microparticles of active ingredient.

According to a particular embodiment, the solid form according to the invention can thus comprise, besides the microparticles of active ingredients, microparticles of viscosifying agent and microparticles of sequestering agent.

### Preparation of the Solid Oral Form

### Core of the Microparticles

The granules forming the core of said microparticles can be obtained by spraying active ingredient in a fluidized bed, optionally with one or more pharmaceutically acceptable excipient(s), such as binding agents, fillers, surfactants, disintegrators, buffering agents, anti-foaming agents onto a support particle, as described previously.

The active ingredient(s) and optional excipients are mixed in solution or dispersed in water or in pharmaceutically acceptable organic solvents with a low boiling point such as ethanol, isopropanol, acetone and mixtures thereof.

### Microparticles

According to a preferred embodiment, the coating arranged on the surface of the modified-release microparticles is obtained by spraying in a fluidized bed, in particular equipped with a Würster and in an upward direction of the spray (bottom spray), of a solution or dispersion containing at least said polymers A and B on the granules obtained above.

Preferably, the polymers A and B and, if appropriate, the plasticizer(s) is(are) sprayed in the solute state i.e. in a solubilized form in a solvent. This solvent is generally constituted by organic solvent(s) mixed or not mixed with water. The organic solvent(s) is/are chosen from the solvents known to a person skilled in the art. By way of example, there can be mentioned acetone, isopropanol, ethanol and mixtures thereof.

The coating thus formed proves homogeneous in terms of composition as opposed to a coating formed by a dispersion of these same polymers, in a mostly aqueous liquid medium, which is not a solvent, or a poor solvent of said polymers A and B

According to a preferred embodiment variant, the sprayed solution contains less than 40% by weight water, in particular less than 30% by weight water and more particularly less than 25% by weight water or even has a water content less than or equal to 10% by weight relative to the total weight of solvents.

### Solid Oral Pharmaceutical Form

According to a particularly preferred embodiment, a solid oral pharmaceutical form according to the invention is a tablet or a gelatin capsule.

In the case of presentation in the form of a gelatin capsule, the microparticles with modified-release of active ingredient, the microparticles of viscosifying agent and the optional microparticles of sequestering agent are mixed beforehand with excipients known to a person skilled in the art such as for example diluents, lubricants or flow agents, as described more precisely hereafter. The mixture obtained is then distributed in gelatin capsules. Alternatively, a sequential method of filling the gelatin capsules can be implemented, the different components being added after one another or in the form of partial mixture(s).

In the case of presentation in the form of a tablet, the microparticles with modified-release of active ingredient, the microparticles of viscosifying agent and the optional microparticles of sequestering agent are mixed beforehand with excipients known to a person skilled in the art such as lubricants or flow agents, diluents or compression agents, as described more precisely hereafter. The mixture is then compressed.

The compression can be carried out according to any conventional method and its implementation is clearly within the competence of a person skilled in the art.

The tablets advantageously possess a significant breaking strength. For example, for a round tablet with a diameter of 12 mm, this hardness can vary from 50 to 500 N, in particular from 60 to 200 N. This hardness can be measured according to the protocol described in the European Pharmacopoeia 6^{th} Edition, Chapter 2.9.8.

According to an embodiment variant, the microparticles with modified-release of active ingredients can be mixed with other modified-release microparticles having different coating compositions or different sizes or also with immediate-release particles of active ingredient.

These are useful general methodologies, which make it possible to produce the solid forms of the invention simply and economically.

The final solid form, in particular in the form of a tablet or gelatin capsule, can, if appropriate, be subjected to additional treatments, according to the techniques and formulae known to a person skilled in the art aimed, for example, at forming on their surface a particular film-coating or coating intended to provide them with additional properties or qualities (colour, appearance, masking of taste, etc.).

According to a particular embodiment, a solid form according to the invention, in particular of tablet or gelatin capsule type, has a charge level of microparticles with modified-release of active ingredients, ranging from 5% to 95% by weight relative to its total weight, in particular from 10% to 90% by weight, and more particularly from 20 to 85% by weight.

### Excipients

As specified previously, a solid oral pharmaceutical form according to the invention is advantageously presented in the form of a tablet or gelatin capsule, containing microparticles of active ingredient as defined above.

The solid oral pharmaceutical form containing the microparticles with modified-release of the active ingredient can thus comprise standard physiologically acceptable excipients which are useful for formulating microparticles within a matrix, for example in the form of a tablet or within a mixture enclosed in a gelatin capsule.

According to a particular embodiment, a solid oral pharmaceutical form according to the invention, of gelatin capsule type, can contain, besides the microparticles with modified-release of active ingredient, microparticles of viscosifying agent and optional microparticles of sequestering agent:
diluents, such as lactose, sucrose, sugar spheres (Suglets® from NP Pharm), microcrystalline cellulose (Avicel® from FMC Biopolymer, Cellet® from Pharmatrans, Ceiphere® from Asahi Kasei), calcium carbonates in crystal form (Omyapure® 35 from Omya) or in the form of particles already formulated with binding agents (Destab® 90 S Ultra 250 from Particle Dynamics or Hubercal® CCG4100 from Huber), the di- and tricalcium phosphates (Dicafos® and Tricafos® from Budenheim), magnesium oxide, calcium phosphate and sulphate;
lubricants or flow agents such as the stearates in particular magnesium stearate, calcium stearate or zinc stearate, stearic acid, glycerol behenate, sodium stearyl fumarate, talc, colloidal silica;
disintegrators, such as the starches and pregelatinized starches (maize starch), carboxymethylcellulose, croscarmellose, crospovidone (grades of Polyplasdone® from ISP, Kollidon® CL from BASF), low substituted hydroxypropylcellulose;
colouring agents or pigments, such as titanium dioxide, calcium sulphate, precipitated calcium carbonate, iron oxides, natural food colouring agents such as caramels, carotenoids, carmine, the chlorophyllins, Rocou (or annatto), the xanthophylls, the anthocyans, betanin, aluminium, and synthetic food colouring agents such as the yellows No. 5 and No. 6, the reds No. 3 and No. 40, the green No. 3 and Emerald green, the blues No. 1 and No. 2;
flavourings, for example strawberry, orange, banana, mint flavourings;
preservatives, such as the parabens, in particular methylparaben, ethylparaben, propylparaben and butylparaben, benzoic acid and its salts (for example sodium benzoate), chlorocresol, sorbic acid and its salts, glycerine;
polyethylene glycol, polyvinyl alcohol, glycerol palmitostearate;
and mixtures thereof.

The choice of these excipients, for an solid oral form of gelatin capsule type, is clearly within the competence of a person skilled in the art.

According to a particular embodiment, a solid form according to the invention of gelatin capsule type can in particular comprise one or more diluent(s) in a content ranging from 0% to 80% by weight, in particular from 0% to 70% by weight, and more particularly from 35% to 65% by weight relative to the total weight of the solid form of gelatin capsule type.

In particular, a solid form according to the invention, of gelatin capsule type, can comprise one or more lubricant(s) or flow agent(s) in a content ranging from 0.1% to 5% by weight, in particular from 0.5% to 2% by weight relative to the total weight of the solid form of gelatin capsule type.

According to a particular embodiment, a solid form according to the invention of gelatin capsule type comprises, besides the microparticles with modified-release of active ingredient defined above, at least one diluent, in particular microcrystalline cellulose, and at least one lubricant or a flow agent, in particular chosen from magnesium stearate, colloidal silica, and mixtures thereof.

In particular, these different excipients are utilized in contents as defined previously.

According to another embodiment, a solid oral pharmaceutical form according to the invention, of tablet type, can contain, besides the microparticles with modified-release of active ingredient, microparticles of viscosifying agent and optional microparticles of sequestering agent:
diluents or compression agents, such as lactose, sucrose, mannitol (grades of Pearlitol® from Roquette, in particular Pearlitol® SD200), xylitol, erythritol, the sorbitols, microcrystalline cellulose (Avicel® from FMC Biopolymer, Cellet® from Pharmatrans or Celphere® from Asahi Kasei), calcium carbonates in crystal form (Omyapure® 35 from Omya) or in the form of particles already formulated with binding agents (Destab® 90 S Ultra 250 from Particle Dynamics or Hubercal® CCG4100 from Huber), the di- and tricalcium phosphates (Dicafos® and Tricafos® from Budenheim), magnesium oxide;
lubricants or flow agents such as the stearates, in particular magnesium stearate, calcium stearate or zinc stearate, stearic acid, glycerol behenate, sodium stearyl fumarate, talc, colloidal silica;
binding agents such as hydroxyethylcellulose, ethylcellulose, hydroxypropylcellulose (Kulcel® from Aqualon-Hercules), hydroxypropylmethylcellulose (or hypromellose) (Methocel® E or K and in particular Methocel K15M from Dow), methylcellulose (Methocel® A15 from Dow), polyvinylpyrrolidone (or povidone) (Plasdone® K29/32 from ISP, Kollidon® 30 from BASF), the vinylpyrrolidone and vinyl acetate copolymers (or copovidone) (Plasdone® S630 from ISP, Kollidon® VA 64 from BASF), polyethylene oxide, the polyalkylene glycols such as for example polyethylene glycol, the dextroses, pregelatinized starches, maltodextrins, polyvinyl alcohol, glycerol palmitostearate;
disintegrators, such as the starches and pregelatinized starches (for example maize starch), carboxymethylcellulose, croscarmellose, crospovidone (grades of Polyplasdone® from ISP, Kollidon® CL from BASF), low substitued hydroxypropylcellulose;
colouring agents or pigments, such as titanium dioxide, calcium sulphate, precipitated calcium carbonate, iron oxides, natural food colouring agents such as caramels, carotenoids, carmine, the chlorophyllins, Rocou (or annatto), the xanthophylls, the anthocyans, betanin, aluminium, and synthetic food colouring agents such as the yellows No. 5 and No. 6, the reds No. 3 and No.40, the green No. 3 and Emerald green, the blues No. 1 and No.2;
flavourings, for example strawberry, orange, banana, mint flavourings;
preservatives, such as the parabens, in particular methylparaben, ethylparaben, propylparaben and butylparaben, benzoic acid and its salts (for example sodium benzoate), chlorocresol, sorbic acid and its salts, glycerine;
and mixtures thereof.

The choice of these excipients for an solid oral form of tablet type is clearly within the competence of a person skilled in the art.

A solid form according to the invention of tablet type can in particular comprise one or more compression agent(s) or diluent(s) in a content ranging from 10% to 80% by weight, in particular from 30% to 75% by weight, and more particularly from 35% to 65% by weight relative to the total weight of the solid form.

A solid form according to the invention, of tablet type, can comprise one or more lubricant(s) or flow agent(s) in a content ranging from 0.1% to 5% by weight, in particular 0.5% to 2% by weight relative to the total weight of the solid form of tablet type.

According to another particular embodiment of the invention, the content of binding agent(s) in a solid form according to the invention of tablet type can range from 0% to 40% by weight, in particular from 0% to 30% by weight, and more particularly from 5 to 20% by weight relative to the total weight of the solid form.

According to a particular embodiment, a solid form according to the invention of tablet type comprises, besides the microparticles with modified-release of active ingredient, defined above, at least one compression agent or a diluent, in particular chosen from microcrystalline cellulose, mannitol and mixtures thereof, and at least one lubricant or a flow agent, in particular chosen from magnesium stearate, colloidal silica and mixtures thereof, and optionally at least one binding agent, in particular chosen from hydroxypropylmethylcellulose and methylcellulose.

In particular, these different excipients are utilized in contents as defined previously.

According to a particular embodiment, a solid form according to the invention, of gelatin capsule or tablet type, comprises less than 1% by weight disintegrator(s) relative to its total weight, and more particularly, contains no disintegrator.

According to yet another particular embodiment, a solid form according to the invention, with respect to the excipients distinct from the modified-release microparticles, contains no waxy compound which is insoluble in the water, and in particular contains no waxes.

The examples and figures which follow are presented by way of illustration and are non-limitative of the field of the invention.

### FIGURES

FIG. 1: Diagrammatic representation of a modified release profile of active ingredient comprising three phases. In acid aqueous medium with a pH less than 4.0, the start of the release of the active ingredient occurs at point A, after a determined residence time. At point B, which corresponds to the increase in the pH, the release of the active ingredient is accelerated.

FIG. 2: Diagram of the microparticles according to the invention with a support particle (1), covered by a layer containing at least one active ingredient (2), itself film-coated by a coating (3) containing at least the polymer A and the polymer B. The relative proportions of these three constitutive elements are not adhered to in this diagram.

FIGS. 3 a and b: Diagram of a solid form, of tablet or gelatin capsule type, containing microparticles with modified-release of active ingredient according to the invention.

FIG. 3a: The solid oral form comprises microparticles with modified-release of active ingredient (1), microparticles of viscosifying agent (2) and one or more pharmaceutically acceptable excipient(s) (3) in the form of a free powder (in the case of a gelatin capsule) or of a solid matrix (in the case of a tablet) in which the microparticles are dispersed.

FIG. 3b: The solid oral form comprises microparticles with modified-release of active ingredient (1), microparticles of viscosifying agent (2), microparticles of sequestering agent (4) and one or more pharmaceutically acceptable excipient(s) (3) in which the microparticles are dispersed.

FIG. 4: Comparative in vitro release profiles, obtained for microparticles of oxymorphone hydrochloride, prepared according to Example 1, in the different dissolution media 0.1N HCl and phosphate buffer at pH 4.5, pH 6.0, pH 6.8 and pH 7.4.

FIG. 5: Photos of the microparticles of oxymorphone hydrochloride, prepared according to Example 1, intact (5a) and crushed (5b) for 50 revolutions with a pestle and mortar, as explained in Example 1.

FIG. 6: Comparative in vitro release profiles, obtained for microparticles of oxymorphone hydrochloride, intact and crushed for 50 revolutions with a pestle and mortar, prepared and crushed according to Example 1, in a 0.1N HCl dissolution medium.

FIG. 7: Comparative in vitro release profiles, obtained for tablets of oxymorphone hydrochloride prepared according to Example 2, and microparticles of oxymorphone hydrochloride prepared according to Example 1, during sequenced exposure for 2 hours in a 0.1N HCl dissolution medium, then phosphate buffer at pH 7.4.

FIG. 8: Comparative in vitro release profiles, obtained for tablets of oxymorphone hydrochloride, intact and crushed for 50 revolutions with a pestle and mortar, prepared and crushed according to Example 2, in a 0.1N HCl dissolution medium.

FIG. 9: Comparative in vitro release profiles, obtained for gelatin capsules of oxymorphone hydrochloride, intact and crushed for 50 revolutions with a pestle and mortar, prepared and crushed according to Example 3, in a 0.1N HCl dissolution medium.

FIG. 10: Comparative in vitro release profiles, obtained for gelatin capsules of oxymorphone hydrochloride, intact and crushed for 50 revolutions with a pestle and mortar, prepared and crushed according to Example 4, in a 0.1N HCl dissolution medium.

FIG. 11: In vitro release profile of microparticles of oxycodone hydrochloride prepared according to Example 5, during sequenced exposure for 2 hours in a 0.1N HCl dissolution medium, then phosphate buffer at pH 7.4.

FIG. 12: Comparative in vitro release profiles, obtained for microparticles of oxycodone hydrochloride, intact and crushed for 50 revolutions with pestle and mortar, prepared and crushed according to Example 5, in a 0.1N HCl dissolution medium.

FIG. 13: Comparative in vitro release profiles, obtained for microparticles of oxycodone hydrochloride prepared according to Example 5, and for gelatin capsules of oxycodone hydrochloride prepared according to Example 6, during sequenced exposure for 2 hours in a 0.1N HCl dissolution medium, then phosphate buffer at pH 7.4.

FIG. 14: Comparative in vitro release profiles, obtained for gelatin capsules of oxycodone hydrochloride intact and crushed for 50 revolutions with pestle and mortar, prepared and crushed according to Example 6, in a 0.1N HCl dissolution medium.

FIG. 15: Comparative in vitro release profiles of microparticles of oxycodone hydrochloride, with a coating rate of 30% and prepared according to Example 7 (not part of the invention) in the different dissolution media phosphate buffer at pH 6.8 and 0.1N HCl.

FIG. 16: Comparative in vitro release profiles, obtained for microparticles of oxycodone hydrochloride with a coating rate of 30% and prepared according to Example 7 (not part of the invention), intact and crushed for 50 revolutions with pestle and mortar, prepared and crushed according to Example 7 (not part of the invention), in a 0.1N HCl dissolution medium.

FIG. 17: In vitro release profile of microparticles of oxycodone hydrochloride with a size strictly higher than 600 μm, prepared according to Example 8 (not part of the invention), during sequenced exposure for 2 hours in a 0.1N HCl dissolution medium, then phosphate buffer at pH 7.5.

FIG. 18: Comparative in vitro release profiles, obtained for microparticles of oxycodone hydrochloride with a size strictly higher than 600 μm, intact and crushed for 50 revolutions with pestle and mortar, prepared and crushed according to Example 8 (not part of the invention), in a 0.1N HCl dissolution medium.

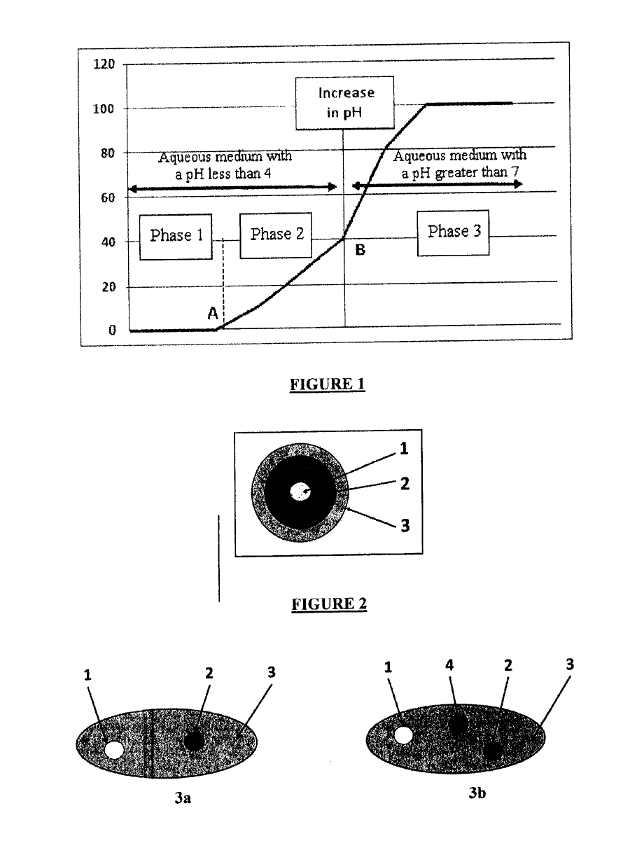

### EXAMPLE 1

Preparation of Microparticles of Oxymorphone Hydrochloride

Phase 1: Preparation of the Granules

1615 g of oxymorphone hydrochloride and 85 g of povidone (Plasdone K29/32 from ISP) are introduced under stirring into a reactor containing 2052.1 g of water and 1105.0 g of ethanol. The solution is heated at 65 ° C. When the oxymorphone hydrochloride crystals and the povidone are dissolved, all of the solution is sprayed onto 300 g of cellulose spheres (Cellet® 90 from Pharmatrans) in a GPCG1.1 fluidized bed in a bottom spray configuration. After spraying, the product obtained is sieved on 80 μm and 250 μm sieves. 1801.9 g of 80 μm to 250 μm granules (which corresponds to the fraction of product having passed through the meshes of the 250 μm sieve and being retained on the 80 μm sieve) are then recovered.

Stage 2: Coating Phase

450 g of granules obtained during phase 1 are coated at ambient temperature, in a GPCG1.1 fluidized bed, with 90 g of a methacrylic acid and ethyl acrylate copolymer (Eudragit® L100-55 from Evonik), 135 g of a methacrylic acid and methyl methacrylate copolymer (Eudragit® S100 from Evonik), 180 g of ethyl cellulose (Ethocel® 20 premium from Dow) and 45 g of triethyl citrate (from Morflex) dissolved in an acetone/isopropanol/water mixture (54/36/10 weight percentage). After spraying, the coated microparticles are recovered. Their volume mean diameter, determined according to the method described in detail hereafter, is 270 μm.

Measurement of the Mean Diameter D(4;3) By Laser Diffraction

The size distribution of the particles is measured by laser diffraction using a Mastersizer® 2000 device from Malvern Instruments equipped with a dry powder sampler of Scirocco 2000 type. Starting from the particle-size distribution measured over a wide range, the equivalent volume mean diameter or D(4;3) is calculated according to the following formula:

D(4;3)=Σ(d⁴)/Σ(d³)

Dissolution Profiles of the Microparticles

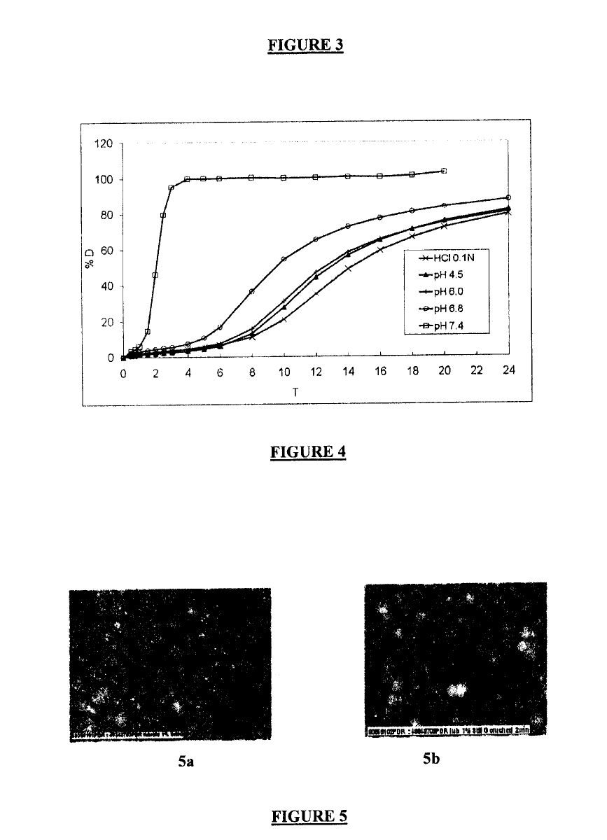

The in vitro dissolution profiles of the microparticles prepared above are measured by UV spectrometry in 900 ml of the dissolution media 0.1 N HCl and phosphate buffer at pH 4.5, pH 6.0, pH 6.8 and pH 7.4, all maintained at 37.0±0.5° C. and stirred with a paddle revolving at 100 rpm. The dissolution profiles obtained for the microparticles in the different media are presented in FIG. 4. The dissolution profiles show an increase in the in vitro release rate of the active ingredient in the phosphate buffer media at pH 6.8 and pH 7.4 relative to the release rates observed in the dissolution media 0.1N HCl, and phosphate buffer at pH4.5 and at pH 6.0.

Crushing of the Microparticles

A fraction of the microparticles obtained during phase 2 and corresponding to a dose of 80 mg of oxymorphone hydrochloride, i.e. approximately 320 mg was crushed using a 250 ml pyrex mortar and a pyrex pestle for 50 revolutions (i.e. approximately 1 minute). The crushing test used is described in detail hereafter.

The photos, taken under a binocular magnifier, of the microparticles before and after crushing are shown in FIGS. 5a and 5b respectively.

The appearance of the microparticles, observed under a binocular magnifier, is the same before and after crushing.

Crushing Test

As previously mentioned, the pestle and mortar technique is used as a crushing test in order to determine the resistance to crushing of the microparticles of active ingredient or of the solid oral pharmaceutical form containing the microparticles of active ingredient according to the invention.

A dose of active ingredient in the form of microparticles or of an intact oral pharmaceutical form (a tablet or the content of a gelatin capsule) is introduced into a 250 ml pyrex mortar and crushed using the pyrex pestle corresponding to the mortar for 50 revolutions, i.e. for approximately 1 minute.

The in vitro release rate of the active ingredient contained in the powder obtained after crushing is then determined during a dissolution test. This test, which is identical to the dissolution test of the microparticles or of the intact oral pharmaceutical form, is carried out according to the method of the European Pharmacopoeia 6^{th} Edition, 6.5 Chapter 2.9.3—Test for dissolution of the solid forms.

The dissolution profiles of the intact and crushed microparticles or of the intact and crushed oral pharmaceutical form are compared: the difference at 0.5 hour between the dissolution profile of the crushed powder and that of the intact formulation corresponds to the proportion of microparticles or of the oral pharmaceutical form damaged after crushing, the remaining proportion corresponding to the proportion of microparticles or oral pharmaceutical form according to the invention which have resisted crushing.

Dissolution Profiles of the Intact And Crushed Particles

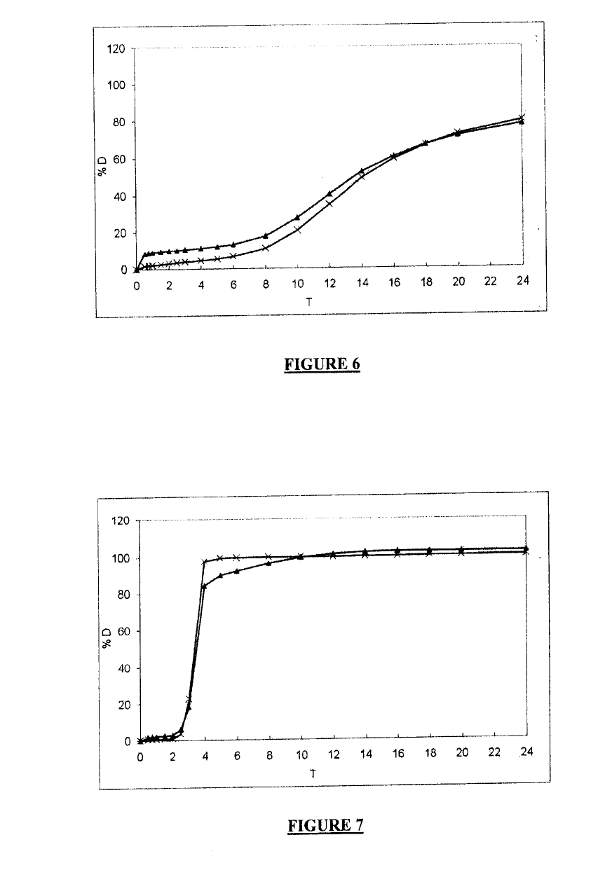

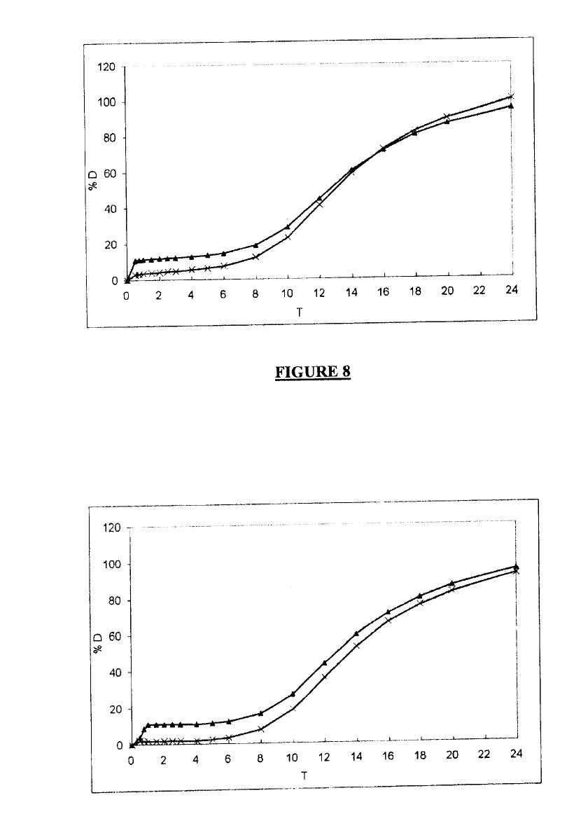

The in vitro dissolution profiles of the intact microparticles, prepared above, and of the same crushed microparticles, are measured by UV spectrometry in 900 ml of a 0.1 N HCl dissolution medium maintained at 37.0±0.5° C. and stirred with a paddle revolving at 100 rpm. The dissolution profiles obtained for the intact ( [symbol] ) and crushed ( [symbol] ) microparticles are compared in FIG. 6. The two dissolution profiles are similar and have a similarity factor according to the European Pharmacopoeia of 62%. It is noted that less than 10% of the microparticles have been damaged. The microparticles which have been subjected to crushing have retained their modified release properties.

### EXAMPLE 2

Preparation of Tablets of Oxymorphone Hydrochloride

Preparation of Tablets

11.0 g of the delayed and modified release microparticles prepared in the previous example (phase 2), are mixed with 8.0 g of polyoxyethylene (Sentry Polyox WSR® 303 from Dow), previously sieved on the 150 μm and 300 μm sieves, the retained fraction being of a size which is comprised between 150 μm and 300 μm), 2.0 g of hypromellose (Methocel® K15M EP from Colorcon), 12.0 g of methyl cellulose (Methocel® A15 LV from Colorcon), 24.0 g of microcrystalline cellulose (Avicel® PH301 from FMC), 24.0 g of mannitol (Pearlitol® SD 200 from Roquette), 40 g of cellulose spheres (from Asahi Kasei) and 1.0 g of magnesium stearate. This mixture is used for the production of round 611 mg tablets with a diameter of 12 mm, using a Korsch XP1 press. The compression force applied to the mixture is 25 kN. The tablets thus produced have a hardness of approximately 97 N.

Dissolution Profiles Under Sequential Exposure Conditions

The in vitro dissolution profile of the tablet prepared above is measured by UV spectrometry in 900 ml of a 0.1 N HCl dissolution medium maintained at 37.0±0.5° C. and stirred with a paddle revolving at 100 rpm for 2 hours then, after adjustment of the pH and salinity of the medium, with phosphate buffer at a pH of 7.4 and 0.05 M of potassium phosphate.

The dissolution profile of the tablet obtained ( [symbol] ) is compared in FIG. 7 with the profile of the intact microparticles ( [symbol] ) prepared according to Example 1. The two dissolution profiles are similar and have a similarity factor according to the European Pharmacopoeia of 58%.

Crushing of the Tablets

A tablet obtained according to Example 2, corresponding to a 20 mg dose of oxymorphone hydrochloride, was crushed using a 250 ml pyrex mortar and a pyrex pestle for 50 revolutions (i.e. approximately 1 minute). The crushing test used is described above.

Dissolution Profiles of the Intact And Crushed Tablets

The in vitro dissolution profiles of the intact tablets, prepared above, and of the same tablets crushed, are measured by UV spectrometry in 900 ml of 0.1 N HCl maintained at 37.0±0.5° C. and stirred with a paddle revolving at 100 rpm. The dissolution profiles obtained for the intact ( [symbol] ) and crushed tablets ( [symbol] ) are compared in FIG. 8. Approximately 10% of the microparticles have been damaged. The other microparticles have retained their modified release profile. The two dissolution profiles are similar and have a similarity factor according to the European Pharmacopoeia of 61%.

In Vitro Test On Extraction For Injection

A tablet of oxymorphone hydrochloride prepared above is crushed using a 250 ml pyrex mortar and a pyrex pestle for 50 revolutions (i.e. approximately 1 minute). 10 ml of tap water are poured onto the powder. The dispersion is then stirred for 10 minutes using a magnetic stirrer. The dispersion is then drawn off over 5 minutes with a 10 ml syringe, through a 27G needle the tip of which is covered by a cotton pellet.

The quantity of liquid drawn off into the syringe is less than 0.1 ml, corresponding to less than 1% of the volume of extraction solvent introduced.

### EXAMPLE 3

Preparation of Gelatin Capsules of Oxymorphone Hydrochloride

Preparation of the Gelatin Capsules

13.8 g of the modified-release microparticles prepared in Example 1 (phase 2), are mixed with 10.0 g of polyoxyethylene (Sentry Polyox WSR® 303 from Dow, previously sieved on 150 μm and 300 μm sieves, the retained fraction being of a size which is comprised between 150 μm and 300 μm), 50.0 g of cellulose spheres (from Asahi Kasei), 0.8 g of colloidal silica (Aerosil® 200 from Evonik) and 0.4 g of magnesium stearate. This mixture is used for the production of gelatin capsules of size 0 containing 300 mg of mixture i.e. a 20 mg dose of oxymorphone hydrochloride.

Crushing of the Gelatin Capsules

The content of a gelatin capsule obtained according to example 3 was crushed using a 250 ml pyrex mortar and a pyrex pestle for 50 revolutions (i.e. approximately 1 minute).

Dissolution Profiles of the Intact And Crushed Gelatin Capsules

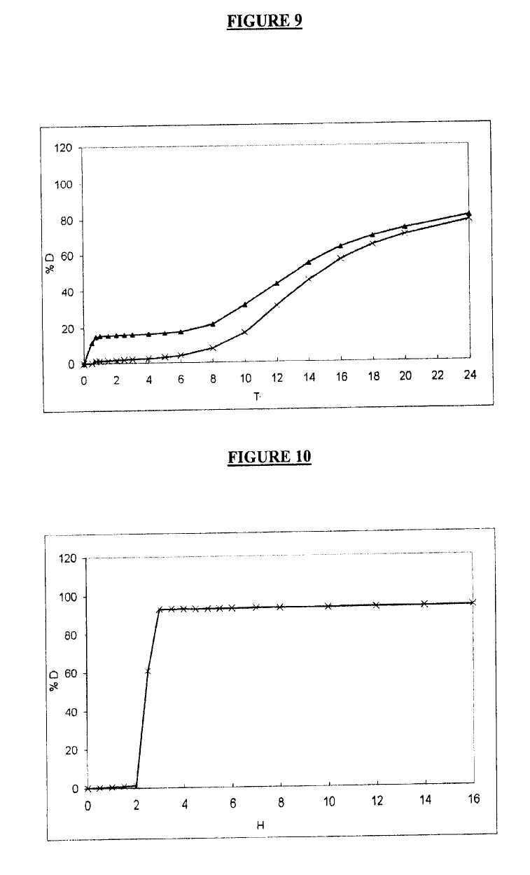

The in vitro dissolution profiles of the intact gelatin capsules, prepared above, and of the crushed content of the same gelatin capsules, are measured by UV spectrometry in 900 ml of a 0,1 N HCl dissolution medium maintained at 37.0±0.5° C. and stirred with a paddle revolving at 100 rpm. The dissolution profiles obtained for the intact gelatin capsules ( [symbol] ) and for the crushed content of the gelatin capsules ( [symbol] ) are compared in FIG. 9. Approximately 10% of the microparticles have been damaged. The other microparticles have retained their modified release profile. The two dissolution profiles are similar and have a similarity factor according to the European Pharmacopoeia of 56%.

In Vitro Test On Extraction For Injection

The content of a gelatin capsule of oxymorphone hydrochloride obtained according to Example 3 is crushed using a 250 ml pyrex mortar and a pyrex pestle for 50 revolutions (i.e. approximately 1 minute). 10 ml of tap water are poured onto the crushed powder. The dispersion is then stirred for 10 minutes using a magnetic stirrer. The dispersion is then drawn off over 5 minutes with a 10 ml syringe, through a 27 G needle the tip of which is covered by a cotton pellet.

The quantity of liquid drawn off into the syringe is less than 0.6 ml, corresponding to less than 6% of the volume of extraction solvent introduced.

### EXAMPLE 4

Preparation of Gelatin Capsules of Oxymorphone Hydrochloride

Preparation of the Gelatin Capsules

13.8 g of the modified-release microparticles prepared in Example 1 (phase 2), are mixed with 10.0 g of polyoxyethylene (Sentry Polyox WSR® 303 from Dow, previously sieved on 150 μm and of 300 μm sieves, the retained fraction being of a size which is comprised between 150 μm and 300 μm), 25.0 g of cation exchange resin (Amberlite® IR69F from Rohm & Haas previously dried, crushed and sieved on 150 μm and 300 μm sieves, the retained fraction being of a size which is comprised between 150 μm and 300 μm), 50.0 g of cellulose spheres (from Asahi Kasei), 1.0 g of colloidal silica (Aerosil® 200 from Evonik) and 0.5 g of magnesium stearate. This mixture is used for the production of gelatin capsules of size 0 containing 401 mg of mixture i.e. a 20 mg dose of oxymorphone hydrochloride.

Crushing of the Gelatin Capsules

The content of a gelatin capsule obtained according to Example 4 was crushed using a 250 ml pyrex mortar and a pyrex pestle for 50 revolutions (i.e. approximately 1 minute).

Dissolution Profiles of the Intact And Crushed Gelatin Capsules

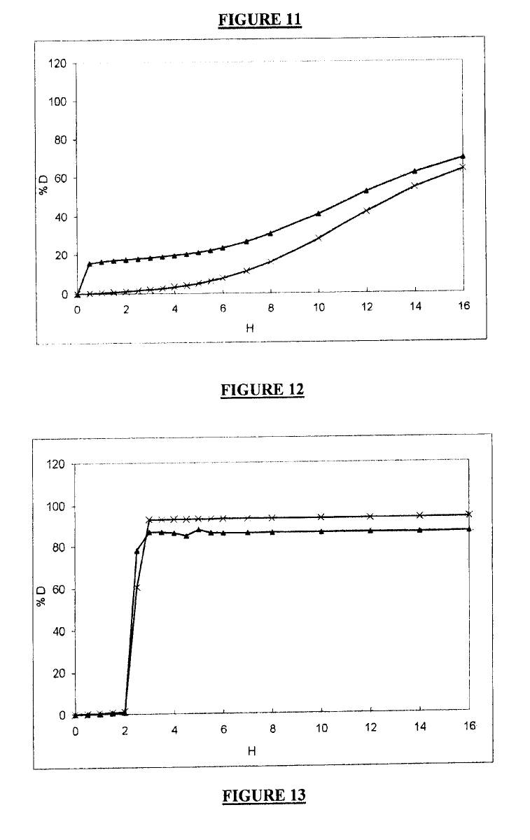

The in vitro dissolution profiles of the intact gelatin capsules, prepared above, and of the crushed content of the same gelatin capsules, are measured by UV spectrometry in 900 ml of a 0.1 N HCl dissolution medium maintained at 37.0±0.5° C. and stirred with a paddle revolving at 100 rpm. The dissolution profiles obtained for the intact gelatin capsules ( [symbol] ) and for the crushed content of the gelatin capsules ( [symbol] ) are compared in FIG. 10. Only 15% of the dose of oxymorphone hydrochloride contained in the gelatin capsules is available immediately. The other microparticles have remained intact and have retained their modified release profile.

In Vitro Test On Extraction For Injection

The content of a gelatin capsule of oxymorphone hydrochloride obtained according to Example 4 is crushed using a 250 ml pyrex mortar and a pyrex pestle for 50 revolutions (i.e. approximately 1 minute). 10 ml of tap water are poured onto the crushed powder. The dispersion is then stirred for 10 minutes using a magnetic stirrer. The dispersion is then drawn off over 5 minutes with a 10 ml syringe, through a 27G needle the tip of which is covered by a cotton pellet.

The quantity of liquid drawn off into the syringe is less than 0.6 ml, corresponding to less than 6% of the volume of extraction solvent introduced.

In Vitro Test On Extraction For Oral Ingestion

The content of a gelatin capsule obtained according to Example 4, corresponding to a 20 mg dose of oxymorphone hydrochloride, was crushed using a 250 ml pyrex mortar and a pyrex pestle for 50 revolutions (i.e. approximately 1 minute). The crushed powder is recovered and introduced into a 125 ml polyethylene bottle into which 100 ml of tap water are poured. The dispersion contained in the polyethylene bottle closed with a threaded stopper is stirred for 7 hours at ambient temperature using an inclined rotating disc at 45 ° C. and at a speed of rotation of 30 rpm then left to rest in the closed polyethylene bottle at ambient temperature. Two samples of 3 ml of the dispersion are taken after 7 hours and after 24 hours of the crushed powder being brought into contact with the 100 ml of extraction liquid, and are then filtered on 0.45 μm Acrodisc® filters and analyzed by HPLC chromatography.

The proportions of oxymorphone hydrochloride dissolved in the extraction liquid (tap water) relative to the 20 mg of crushed oxymorphone hydrochloride introduced into the 100 ml of extraction liquid are presented in the table below.

After 24 hours of dispersion of the content of the gelatin capsule, prepared and crushed according to the previous stages, only 12% of the dose of oxymorphone hydrochloride contained in the gelatin capsule (20 mg), i.e. 2.4 mg of oxymorphone hydrochloride, is available immediately.

| | Proportions of oxymorphone hydrochloride |
|---|---|
| | dissolved in the extraction liquid relative to |
| Time of the Sampling | the dose introduced |
| 7 hours | 8.1% |
| 24 hours | 12.0% |

### Example 5

Preparation of Microparticles of Oxycodone Hydrochloride

Phase 1: Preparation of the Granules

1615.0 g of oxycodone hydrochloride and 85.0 g of povidone (Plasdone® K29/32 from ISP) are introduced under stirring into a reactor containing 2052.1 g of water and 1105.0 g of ethanol. The solution is heated at 65 ° C. When the oxycodone hydrochloride crystals and the povidone are dissolved, all of the solution is sprayed onto 300.0 g of cellulose spheres (Cellet® 90 from Pharmatrans) in a GPCG1.1 fluidized bed in a bottom spray configuration.

After spraying, the product obtained is sieved on 80 μm and 250 μm sieves. 2054.6 g of 80 μm to 250 μm granules (which corresponds to the fraction of product having passed through the meshes of the 250 gm sieve and being retained on the 80 μm sieve) are then recovered.

Phase 2: Coating Phase

400.0 g of granules obtained during phase 1 are coated at ambient temperature, in a GPCG1.1 fluidized bed, with 119.99 g of a methacrylic acid and ethyl acrylate copolymer (Eudragit® L100-55 from Evonik), 80.01 g of a methacrylic acid and methyl methacrylate copolymer (Eudragit® S100 from Evonik), 160.02 g of ethylcellulose (Ethocel® 20 premium from Dow) and 40.02 g of triethyl citrate (Citrofol AI from Jungbunzlauer) dissolved in a mixture of 2484.0 g of acetone, 1656.0 g of isopropanol and 460.0 g of water.

After spraying 3333 g of coating solution, a sample of 11.5 g of particles is taken. The coating rate of the sampled microparticles is 40%. The volume mean diameter of the sampled microparticles, determined by laser diffraction according to the method previously described, is 275 μm.

Dissolution Profiles Under Sequential Exposure Conditions

The in vitro dissolution profile of the microparticles of oxycodone hydrochloride, prepared above, is measured by UV spectrometry in 900 ml of a 0.1 N HCl for 2 hours then, after adjustment of the pH and salinity of the medium, at a pH of 7.4 and 0.05 M of potassium phosphate, maintained at 37.0±0.5° C. and stirred with a paddle revolving at 100 rpm.

The dissolution profile obtained is presented in FIG. 11. The microparticles of oxycodone hydrochloride prepared show a release profile depending from time and from the pH of the surrounding medium.

Crushing of the Microparticles

The amount of microparticles obtained during phase 2 and corresponding to a dose of 80 mg of oxycodone hydrochloride, i.e. approximately 175 mg was crushed using a 250 ml pyrex mortar and a pyrex pestle for 50 revolutions (i.e. approximately 1 minute). The crushing test used is described above.

Dissolution Profiles of the Intact And Crushed Particles

The in vitro dissolution profiles of the intact microparticles, prepared above during phase 2, and of the same crushed microparticles, are measured by UV spectrometry in 900 ml of a 0.1 N HCl maintained at 37.0±0.5° C. and stirred with a paddle revolving at 100 rpm. The dissolution profiles obtained for the intact ( [symbol] ) and crushed ( [symbol] ) microparticles are compared in FIG. 12.

It is noted that only 15% of the microparticles have been damaged. The other microparticles have retained their modified release properties.

### Example 6

Preparation of Gelatin Capsules of Oxycodone Hydrochloride

Preparation of the Gelatin Capsules

1.730 g of the modified-release microparticles prepared in Example 5 (phase 2) are mixed with 0.400 g of polyoxyethylene (Sentry Polyox WSR® 303 from Dow, previously sieved on 150 μm and of 300 μm sieves, the retained fraction having a size comprised between 150 μm and 300 μm), 1.007 g of cation exchange resin (Amberlite® IR69F from Rohm & Haas previously dried, crushed and sieved on 150 μm and 300 μm sieves, the retained fraction having a size comprised between 150 μm and 300 μm), 0.035 g of colloidal silica (Aerosil® 200 from Evonik) and 0.016 g of magnesium stearate. This mixture is used for the production of gelatin capsules of size 0 containing 319 mg of mixture i.e. an 80 mg dose of oxycodone hydrochloride.

Dissolution Profiles Under Sequential Exposure Conditions

The in vitro dissolution profile of the gelatin capsules of oxycodone hydrochloride, prepared above, is measured by UV spectrometry in 900 ml of a 0.1 N HCl for 2 hours then, after adjustment of the pH and salinity of the medium, at a pH of 7.5 and 0.05 M of potassium phosphate, maintained at 37.0±0.5° C. and stirred with a paddle revolving at 100 rpm.

The dissolution profile of the gelatin capsules obtained ( [symbol] ) is compared in FIG. 13 with the profile of the intact microparticles ( [symbol] ) prepared according to Example 5. The two dissolution profiles are similar and have a similarity factor according to the European Pharmacopoeia of 58%.

Crushing of the Gelatin Capsules

The content of a gelatin capsule obtained above was crushed using a 250 ml pyrex mortar and a pyrex pestle for 50 revolutions (i.e. approximately 1 minute).

Dissolution Profiles of the Intact And Crushed Gelatin Capsules

The in vitro dissolution profiles of the intact gelatin capsules, prepared above, and of the crushed content of the same gelatin capsules, are measured by UV spectrometry in 900 ml of a 0.1 N HCl for 2 hours then, after adjustment of the pH and salinity of the medium, with phosphate buffer at a pH of 7.4 and 0.05 M of potassium phosphate, maintained at 37.0±0.5° C. and stirred with a paddle revolving at 100 rpm. The dissolution profiles obtained for the intact gelatin capsules ( [symbol] ) and for the crushed content of the gelatin capsules ( [symbol] ) are compared in FIG. 14.

It is noted that only 10% of the microparticles have been damaged during the crushing. The other microparticles have retained their modified release profile.

In Vitro Test of Extraction For Injection

The content of a gelatin capsule of oxycodone hydrochloride obtained according to Example 6 is crushed using a 250 ml pyrex mortar and a pyrex pestle for 50 revolutions (i.e. approximately 1 minute). 10 ml of tap water are poured onto the crushed powder. The dispersion is then stirred for 10 minutes using a magnetic stirrer. The dispersion is then drawn off over 5 minutes with a 10 ml syringe, through a 27G needle the tip of which is covered by a cotton pellet.

The quantity of liquid drawn off into the syringe is less than 0.2 ml, corresponding to less than 2% of the volume of extraction solvent introduced.

### EXAMPLE 7

### Not Part of the Invention

Preparation of Microparticles of Oxycodone Hydrochloride With A Coating Rate of 30%

2143.0 g of coating solution obtained during phase 2 of Example 5, are sprayed onto 400.0 g of granules obtained during phase 1 of Example 5. A sample of 9.0 g of particles is taken. The coating rate of sampled microparticles is 30%. The volume mean diameter of sampled microparticles is 263 μm.

Dissolution Profiles of Microparticles

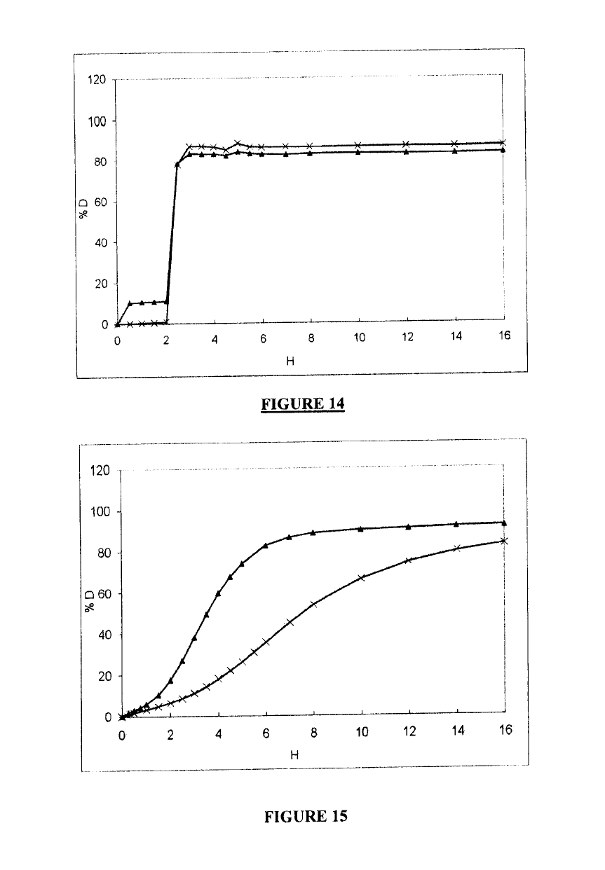

The in vitro dissolution profile of the microparticles of oxycodone hydrochloride, prepared above, is measured by UV spectrometry in 900 ml of 0.1 N HCl and in 900 ml of 0.05 M potassium phosphate buffer at a pH of 6.8, maintained at 37.0±0.5° C. and stirred with a paddle revolving at 100 rpm. The dissolution profiles obtained are presented in FIG. 15.

The microparticles of oxycodone hydrochloride prepared with a coating rate of 30% do have an accelerated release profile in the medium at a pH of 6.8 ( [symbol] ) compared to the one obtained in 0.1N HCl ( [symbol] ).

Crushing of the Microparticles

Approximately 142 mg of microparticles with a coating rate of 30%, corresponding to a dose of 80 mg of oxycodone hydrochloride, were crushed using a 250 ml pyrex mortar and a pyrex pestle for 50 revolutions (i.e. approximately 1 minute).

Dissolution Profiles of the Intact And Crushed Particles

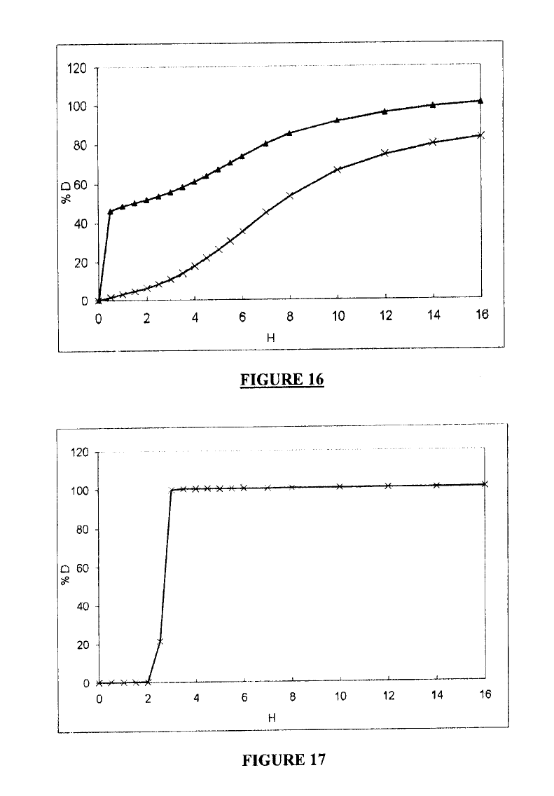

The in vitro dissolution profiles of the intact microparticles, prepared above, and of the same crushed microparticles, are measured by UV spectrometry in 900 ml of 0.1 N HCl maintained at 37.0±0.5° C. and stirred with a paddle revolving at 100 rpm. The dissolution profiles obtained for the intact ( [symbol] ) and crushed ( [symbol] ) microparticles are compared in FIG. 16.

It is noted that 67% of the microparticles were damaged during the crushing and have not retained their modified release properties.

### EXAMPLE 8

### Not Part of the Invention

Preparation of Microparticles of Oxycodone Hydrochloride Having A Size Strictly Greater Than 600 μm.

Phase 1: Preparation of the Granules

137.3 g of oxycodone hydrochloride and 7.2 g of povidone (Plasdone® K29/32 from ISP) are introduced under stirring into a reactor containing 174.4 g of water and 93.9 g of ethanol. The solution is heated at 65° C. When the oxycodone hydrochloride crystals and the povidone are dissolved, all the solution is sprayed onto 25.5 g of cellulose spheres (Celphere CP203 from Asahi Kasei) in a MinGlatt 8008 fluidized bed in a bottom spray configuration.

After spraying, 157.1 g of granules are recovered.

Phase 2: Coating Phase

40.0 g of granules obtained during phase 1 are coated at ambient temperature, in a GPCG1.1 fluidized bed, with 14.67 g of a methacrylic acid and ethyl acrylate copolymer (Eudragit® L100-55 from Evonik), 2.60 g of a methacrylic acid and methyl methacrylate copolymer (Eudragit® S100 from Evonik), 6.66 g of ethylcellulose (Ethocel® 20 premium from Dow) and 2.67 g of triethyl citrate (Citrofol AI from Jungbunzlauer) dissolved in a mixture of 165.6 g of acetone, 110.4 g of isopropanol and 30.70 g of water.

After spraying, coated microparticles are recovered. The volume mean diameter of the recovered microparticles is 666 μm.

Dissolution Profiles Under Sequential Exposure Conditions

The in vitro dissolution profile of the microparticles of oxycodone hydrochloride, prepared above, is measured by UV spectrometry in 900 ml of 0.1 N HCl for 2 hours then, after adjustment of the pH and salinity of the medium, at a pH of 7.5 and 0.05 M of potassium phosphate, maintained at 37.0±0.5° C. and stirred with a paddle revolving at 100 rpm.

The dissolution profile obtained is presented in FIG. 17. The microparticles of oxycodone hydrochloride with a size greater than 600 μm show a release profile depending from time and from the pH of the surrounding medium.

Crushing of the Microparticles

Approximately 174 mg of microparticles prepared during the phase 2, corresponding to a dose of 80 mg of oxycodone hydrochloride, were crushed using a 250 ml pyrex mortar and a pyrex pestle for 50 revolutions (i.e. approximately 1 minute).

Dissolution Profiles of the Intact And Crushed Particles

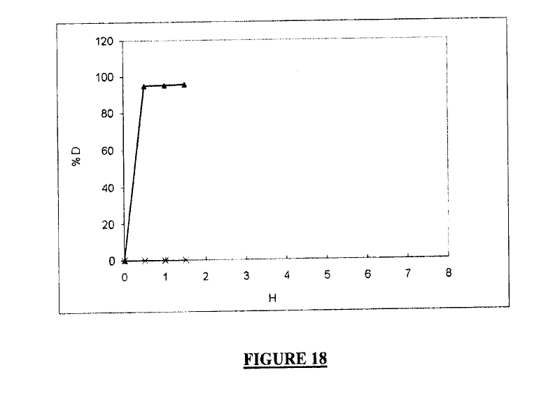

The in vitro dissolution profiles of the intact microparticles, prepared above during the phase 2, and of the same crushed microparticles, are measured by UV spectrometry in 900 ml of 0.1 N HCl maintained at 37.0±0.5° C. and stirred with a paddle revolving at 100 rpm. The dissolution profiles obtained for the intact ( [symbol] ) and crushed ( [symbol] ) microparticles are compared in FIG. 18.

From the first samplings of the dissolution test, i.e. from 30 min in the acid medium, the crushed microparticles have released 100% of the dose of oxycodone hydrochloride initially contained in the microparticles.

All microparticles with a size greater than 600 μm were damaged during the crushing. They have not retained their modified release properties after crushing.

1. Solid oral pharmaceutical form with modified release of at least one active ingredient, containing at least microparticles containing said active ingredient and at least one viscosifying agent in a form isolated from said microparticles of active ingredient, characterized in that said microparticles possess an average diameter ranging from 100 to 600 μm, and are formed by a core containing at least said active ingredient and coated with at least one coating layer,
said core being formed by a support particle covered by a layer comprising at least said active ingredient,
said coating layer being formed by a material composed of at least:
25 to 70% by weight relative to the total weight of said coating, of at least one polymer A which is insoluble in water,
30 to 75% by weight relative to the total weight of said coating, of at least one polymer B which is insoluble in water below pH 5 and soluble in water above pH 7, and
0 to 25% by weight relative to the total weight of said coating, of at least one plasticizer,
said polymers A and B being in a polymer(s) B/polymer(s) A weight ratio comprised between 0.25 and 4, and
said coating layer representing at least 35% by weight, relative to the total weight of said microparticle.

2. Solid form according to claim 1, in which said microparticles of active ingredient are resistant to crushing.

3. Solid form according to claim 1, in which the coating of said microparticles of active ingredient contains less than 30% by weight of talc, relative to the total weight of said coating, preferably less than 20% by weight, in particular less than 10% by weight, more particularly less than 5% by weight, and even totally free of talc.

4. Solid form according to claim 1, in which the coating of the microparticles is composed of a single layer formed by said material.

5. Solid form according to claim 1, in which the coating arranged on the surface of the microparticles is present at a coating level ranging from 35 to 60% by weight by weight relative to the total weight of said microparticle.

6. Solid form according to claim 1, in which the coating arranged on the surface of the microparticles is obtained by spraying in a fluidized bed, of a solution containing at least said polymers A and B on granules obtained by the application to the surface of a support particle of a layer comprising at least said active ingredient.

7. Solid form according to claim 1, in which the polymer A is chosen from ethylcellulose, cellulose acetate butyrate, cellulose acetate, ammonio (meth)acrylate copolymers, poly(meth)acrylic acid esters, and mixtures thereof.

8. Solid form according to claim 1, in which the coating of the microparticles contains 30 to 65% by weight of polymer(s) A relative to its total weight.

9. Solid form according to claim 1, in which the polymer B is chosen from the methacrylic acid and methyl methacrylate copolymer(s), the methacrylic acid and ethyl acrylate copolymer(s) and mixtures thereof.

10. Solid form according to claim 1, in which the coating of the microparticles contains 30 to 70% by weight of polymer(s) B relative to its total weight.

11. Solid form according to claim 1, in which the coating of the microparticles is formed by at least one mixture comprising, as polymer A, at least ethylcellulose or cellulose acetate butyrate or ammonio (meth)acrylate copolymer or a mixture thereof, with, as polymer B, at least one methacrylic acid and ethyl acrylate copolymer or a methacrylic acid and methyl methacrylate copolymer or a mixture thereof.

12. Solid form according to claim 1, in which the coating comprises the polymers A and B in a polymer(s) B/polymer(s) A weight ratio comprised between 0.3 and 4.

13. Solid form according to claim 1, in which said support particle possesses an average diameter less than or equal to 300 μm.

14. Solid form according to claim 1, in which said coated microparticles possess an average diameter ranging from 150 to 350 μm.

15. Solid form according to claim 1, in which the viscosifying agent is in the form of microparticles, distinct from the microparticles with modified-release of active ingredient.

16. Solid form according to claim 1, characterized in that the viscosifying agent is chosen from:
the polyacrylic acids, in particular the carbomers,
the polyalkylene glycols, for example the polyethylene glycols,
the polyalkylene oxides, for example the polyethylene oxides or polyoxyethylene,
the polyvinylpyrrolidones,
the gelatins,
the polysaccharides, preferably chosen from sodium alginate, the pectins, guar gum, the xanthans, carrageenans, gellans, hydroxypropylcellulose, hydroxypropylmethylcellulose, methylcellulose, hydroxyethylcellulose and carboxymethylcellulose,
and mixtures thereof.

17. Solid form according to claim 1, in which the viscosifying agent is a polyoxyethylene, in particular possessing a high molecular weight, and more particularly having an average molecular weight ranging from 1 million g/mole to approximately 8 million g/mole.

18. Solid form according to claim 1, in which the active ingredient is chosen from the amphetamines, analgesics, anorexigens, antidepressants, antiepileptics, antiparkinsonians, anxiolytics, barbiturates, benzodiazepines, hypnotics, narcotics in particular the opioids, the neuroleptics, psychostimulants and psychotropics.

19. Solid form according to claim 1, characterized in that the active ingredient is a narcotic, more particularly chosen from oxycodone, oxymorphone, hydromorphone, hydrocodone, tramadol and their pharmaceutically acceptable salts.

20. Solid form according to claim 1, characterized in that it is presented in the form of a tablet or gelatin capsule.

21. Solid form according to claim 1, characterized in that it comprises at least one sequestering agent in the form of microparticles distinct from microparticles of active ingredient.

22. Solid form according to claim 21, in which the sequestering agent is chosen from:
sodium dodecyl sulphate or sodium docusate;
quaternary ammonium salts, in particular tetradecyl trimethyl ammonium bromide or benzethonium chloride;
strongly acid cation exchange resins when the active ingredient in solution is cationic, or strongly basic anion exchange resins when the active ingredient in solution is anionic, according to the polarity of the active ingredient, and mixtures thereof,
and in particular, when the active ingredient in solution is in cationic form, from:
strongly acid cation exchange resins, such as the sulfonated copolymers of styrene and divinylbenzene, and
weakly acid cation exchange resins, such as the cross-linked copolymers of methacrylic acid and divinylbenzene or their salts.
